(19) United States
(12) Patent Application Publication
(10) Pub. No.: US 2026/0053728 A1
(43) Pub. Date: Feb. 26, 2026

(54) Topical Hair Growth Oil Compound

(71) Applicant: Sherita Hubbard, Dublin, GA (US)

(72) Inventor: Sherita Hubbard, Dublin, GA (US)

(21) Appl. No.: 19/185,819

(22) Filed: Apr. 22, 2025

Related U.S. Application Data
(60) Provisional application No. 63/685,892, filed on Aug. 22, 2024.

(51) Int. Cl. A61K 8/9789; A61K 8/40; A61K 8/49; A61K 8/65; A61K 8/67; A61K 8/73; A61K 8/92; A61K 8/9794; A61Q 7/00

(52) U.S. Cl. CPC A61K 8/9789; A61K 8/40; A61K 8/4953; A61K 8/65; A61K 8/673; A61K 8/678; A61K 8/735; A61K 8/922; A61K 8/9794; A61Q 7/00

(57) ABSTRACT

A topical hair growth oil compound is provided. The device is comprised of a topical hair growth oil compound formulated to enhance hair growth, strengthen strands, and improve scalp health through a blend of natural oils, botanical extracts, and herbal components. The formulation comprises a primary oil mixture of olive oil, coconut oil, and vitamin E oil, which provide nourishment, hydration, and antioxidant benefits. Infused with dried rosemary and thyme for scalp stimulation and antimicrobial properties, the compound also incorporates chebe powder, including lavender croton, vegetable oil, resin, musk ambrette, prunus mahaleb, and clove to reinforce hair structure and moisture retention. Additional ingredients such as peppermint oil, tea tree oil, Jamaican black castor oil, silk protein, collagen, hyaluronic acid, aloe vera, biotin, and caffeine may be incorporated to address specific hair concerns, including strengthening, hydration, and accelerated growth.

### CROSS-REFERENCE TO RELATED APPLICATION

The present application claims priority to, and the benefit of, U.S. Provisional Application No. 63/685,892, which was filed on Aug. 22, 2024, and is incorporated herein by reference in its entirety.

### FIELD OF THE INVENTION

The present invention relates generally to the field of hair growth compounds. More specifically, the present invention relates to a topical hair growth oil compound formulated with natural oils, botanical extracts, and herbal components to promote hair growth, strengthen strands, and improve scalp health. Accordingly, the present disclosure makes specific reference thereto. Nonetheless, it is to be appreciated that aspects of the present invention are also equally applicable to other like applications, devices, and methods of manufacture.

### BACKGROUND

Repeated application of wigs or tension hairstyles, such as braids, can lead to hair damage over time. Without proper care, natural hair may become brittle and break due to the constant strain placed on the hair shaft and follicles. Hair growth may also be stunted if the scalp lacks adequate hydration and nourishment. In many cases, individuals who frequently wear protective styles experience dryness and hair loss, particularly if the follicles are pulled out or the hair is subjected to excessive friction. Scalp conditions such as alopecia, dandruff, and irritation may develop if the hair and scalp are not properly maintained. Moreover, many hair products fail to provide a balanced composition of moisture, strengthening agents, and bioactive compounds necessary for optimal regrowth. As a result, individuals seeking to protect and restore their hair often struggle to find a reliable solution that effectively promotes hair health.

Therefore, there exists a long-felt need in the art for a topical hair growth oil compound that provides users with a moisturizing treatment to nourish the scalp and hair. There also exists a long-felt need in the art for a topical hair growth oil compound that utilizes a combination of vitamins, oils, and minerals to enhance hair strength and regrowth. Moreover, there exists a long-felt need in the art for a topical hair growth oil compound that supports individuals who wear wigs or tension hairstyles by reducing the risk of breakage, dryness, and hair loss.

The subject matter disclosed and claimed herein, in one embodiment thereof, comprises a topical hair growth oil compound. The device is comprised of a topical hair growth oil compound formulated to enhance hair growth, strengthen strands, and improve scalp health through a blend of natural oils, botanical extracts, and herbal components. The formulation comprises a primary oil mixture of olive oil, coconut oil, and vitamin E oil, which provide nourishment, hydration, and antioxidant benefits. Infused with dried rosemary and thyme for scalp stimulation and antimicrobial properties, the compound also incorporates chebe powder, including lavender croton, vegetable oil, resin, musk ambrette, prunus mahaleb, and clove to reinforce hair structure and moisture retention. The preparation method involves an infusion process, followed by filtration and transfer into application bottles for user convenience. Additional ingredients such as peppermint oil, tea tree oil, Jamaican black castor oil, silk protein, collagen, hyaluronic acid, aloe vera, biotin, and caffeine may be incorporated to address specific hair concerns, including strengthening, hydration, and accelerated growth. The application method involves massaging the oil into the scalp and leaving it for an extended period before washing or using as a leave-in treatment. The adaptable formulation and specialized variations ensure comprehensive scalp nourishment, follicle stimulation, and hair care benefits.

In this manner, the topical hair growth oil compound of the present invention accomplishes all the foregoing objectives and provides a topical hair compound designed to promote hair growth, strengthen hair strands, and improve scalp health. The blend of natural oils, botanical extracts, and bioactive ingredients ensures that the scalp remains hydrated and nourished. By incorporating olive oil, coconut oil, and vitamin E, the compound provides essential moisture and antioxidants to reduce oxidative stress and improve scalp circulation. The inclusion of chebe powder, rosemary, and thyme enhances follicular activity while reducing microbial buildup, further supporting hair regrowth. Additionally, the formulation can be tailored with specialized blends to address specific hair care concerns, such as strengthening fragile hair, improving moisture retention, or stimulating accelerated hair growth.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a topical hair growth oil compound. The topical hair growth oil compound is a scalp treatment formulated to promote hair growth, strengthen hair strands, and improve scalp health. The composition is an oil-based infusion comprising natural oils, botanical extracts, and powdered herbal components.

The formulation includes a primary oil mixture, an herbal infusion, and a botanical powder blend. Olive oil serves as the primary carrier oil, providing nourishment, moisture retention, and antioxidant properties. Coconut oil enhances scalp hydration, improves hair elasticity, and has antimicrobial properties. Vitamin E oil functions as an antioxidant, supporting scalp health and reducing oxidative stress. Dried rosemary herb stimulates circulation and hair follicle activity, while dried thyme herb provides antibacterial and antifungal benefits.

Chebe powder is incorporated to strengthen hair and improve length retention. The chebe powder blend includes lavender croton for moisture retention, vegetable oil for infusion facilitation, and resin for antimicrobial properties and enhanced shine. Musk ambrette acts as a conditioning agent, prunus mahaleb supports scalp health, and clove provides antifungal benefits and improves scalp circulation.

A method of preparing the topical hair growth oil compound involves combining olive oil, coconut oil, and vitamin E oil to form an oil mixture. Dried rosemary and thyme herbs are added to release active compounds, followed by the introduction of chebe powder, comprising lavender croton, vegetable oil, resin, musk ambrette, prunus mahaleb, and clove. The mixture is sealed and infused at room temperature for a preferred period of seven days, then shaken to redistribute settled components. The infused oil is filtered to remove excess particulates and transferred into application bottles. The formulation can be adjusted based on specific needs or production requirements.

Additional essential oils and bioactive ingredients may be incorporated to enhance hair growth and scalp nourishment. Peppermint oil increases circulation, tea tree oil provides antimicrobial effects, Jamaican black castor oil strengthens hair roots, and silk protein or collagen reinforces hair structure. The formulation can also be tailored for specific hair care needs. A strengthening blend includes silk protein and collagen for fragile hair. A moisture lock blend incorporates hyaluronic acid or aloe vera for hydration. An ultra-growth blend integrates biotin and caffeine to stimulate hair follicles.

A method of using the topical hair growth oil compound involves dispensing a measured amount onto the fingertips or scalp, massaging it in circular motions to ensure even distribution and stimulate circulation, and leaving it on for an extended period before washing out or using as a leave-in treatment.

Accordingly, the topical hair growth oil compound of the present invention is particularly advantageous as it provides a topical hair compound designed to promote hair growth, strengthen hair strands, and improve scalp health. The blend of natural oils, botanical extracts, and bioactive ingredients ensures that the scalp remains hydrated and nourished. By incorporating olive oil, coconut oil, and vitamin E, the compound provides essential moisture and antioxidants to reduce oxidative stress and improve scalp circulation. The inclusion of chebe powder, rosemary, and thyme enhances follicular activity while reducing microbial buildup, further supporting hair regrowth. Additionally, the formulation can be tailored with specialized blends to address specific hair care concerns, such as strengthening fragile hair, improving moisture retention, or stimulating accelerated hair growth.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a block diagram view of components of one potential embodiment of a topical hair growth oil compound of the present invention in accordance with the disclosed architecture;

FIG. 2 illustrates a flowchart of a method of making one potential embodiment of a topical hair growth oil compound of the present invention in accordance with the disclosed architecture; and

FIG. 3 illustrates a flowchart of a method of using one potential embodiment of a topical hair growth oil compound of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a topical hair growth oil compound that provides users with a moisturizing treatment to nourish the scalp and hair. There also exists a long-felt need in the art for a topical hair growth oil compound that utilizes a combination of vitamins, oils, and minerals to enhance hair strength and regrowth. Moreover, there exists a long-felt need in the art for a topical hair growth oil compound that supports individuals who wear wigs or tension hairstyles by reducing the risk of breakage, dryness, and hair loss.

The present invention, in one exemplary embodiment, is comprised of a topical hair growth oil compound. The topical hair growth oil compound is a scalp treatment designed to promote hair growth, strengthen hair strands, and enhance scalp health via natural oils, botanical extracts, and powdered herbal components. The formulation is comprised of a primary oil mixture, an herbal infusion, and a botanical powder blend. Olive oil acts as the primary carrier oil, providing nourishment, moisture retention, and antioxidant benefits. Coconut oil supports scalp hydration, improves hair elasticity, and possesses antimicrobial properties. Vitamin E oil serves as an antioxidant, promoting scalp health and minimizing oxidative stress. Dried rosemary herb stimulates circulation and follicular activity, while dried thyme herb offers antibacterial and antifungal properties.

Chebe powder is included to reinforce hair strength and aid in length retention. The chebe powder blend contains lavender croton for moisture retention, vegetable oil to facilitate infusion, and resin for antimicrobial properties and enhanced shine. Musk ambrette functions as a conditioning agent, prunus mahaleb contributes to scalp health, and clove provides antifungal benefits while improving circulation.

The preparation method involves combining olive oil, coconut oil, and vitamin E oil to create an oil mixture. Dried rosemary and thyme herbs are then added to release active compounds, followed by the incorporation of chebe powder, which includes lavender croton, vegetable oil, resin, musk ambrette, prunus mahaleb, and clove. The mixture is sealed and left to infuse at room temperature for a preferred duration of seven days, then shaken to redistribute settled components. After the infusion period, the oil is filtered to remove excess particulates and transferred into application bottles. The formulation may be adjusted to accommodate specific requirements or production needs.

Essential oils and bioactive ingredients may be added to further enhance hair growth and scalp nourishment. Peppermint oil boosts circulation, tea tree oil provides antimicrobial benefits, Jamaican black castor oil strengthens hair roots, and silk protein or collagen reinforces hair structure. The formulation may also be customized for targeted hair care needs. A strengthening blend incorporates silk protein and collagen for fragile hair, a moisture lock blend includes hyaluronic acid or aloe vera for hydration, and an ultra-growth blend combines biotin and caffeine to stimulate hair follicles.

The method of application involves dispensing a measured amount onto the fingertips or directly onto the scalp, massaging in circular motions for even distribution and improved circulation, and allowing the oil to remain for an extended period before washing out or leaving in as a treatment.

As a result, the topical hair growth oil compound provides a specialized formulation aimed at promoting hair growth, strengthening strands, and improving scalp health. The combination of natural oils, botanical extracts, and bioactive ingredients ensures hydration and nourishment while reducing oxidative stress and enhancing circulation. The inclusion of chebe powder, rosemary, and thyme supports follicular activity and minimizes microbial buildup, further aiding hair regrowth. Additionally, the formulation offers adaptability through specialized blends designed to meet various hair care concerns, such as strengthening, moisture retention, and accelerated growth stimulation.

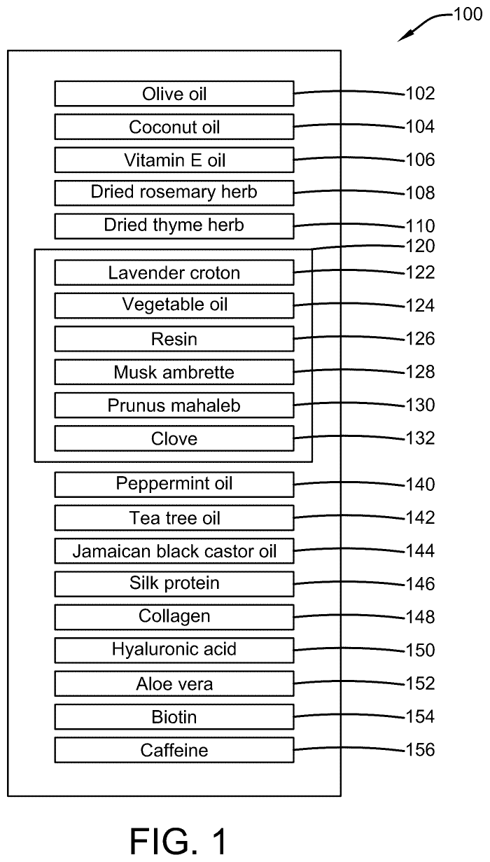

Referring initially to the drawings, FIG. 1 illustrates a block diagram view of components of one potential embodiment of a topical hair growth oil compound 100 of the present invention in accordance with the disclosed architecture. The topical hair growth oil compound 100 is a scalp treatment formulated to promote hair growth, strengthen hair strands, and improve scalp health. The composition is an oil-based infusion comprising multiple natural oils, botanical extracts, and powdered herbal components. The formulation is designed for topical application, with recommended usage twice per week to optimize effectiveness.

The topical hair growth oil compound is comprised of a primary oil mixture, an herbal infusion, and a finely ground botanical powder blend. The base formulation includes between 40-60% olive oil 102, which serves as the primary carrier oil and provides nourishment, moisture retention, and antioxidant properties. 20-40% Coconut oil 104 is included and enhances scalp hydration, improves hair elasticity, and possesses antimicrobial properties. 1-30% Vitamin E oil 106 is included and functions as an antioxidant to support scalp health and reduce oxidative stress on hair follicles. 0.5-5% Dried rosemary herb 108 is included due to its ursolic acid and rosmarinic acid content, which stimulate circulation to the scalp and support hair follicle activity. 0.5-5% Dried thyme herb 110 is included to provide antibacterial and antifungal benefits while promoting hair follicle stimulation.

0.5-5% Chebe powder 120 is incorporated into the formulation to strengthen hair and improve length retention. As seen in FIG. 1, the chebe powder 120 blend comprises lavender croton 122 for its ability to retain moisture and fortify hair strands. Vegetable oil 124 is included to facilitate the infusion process and help distribute active compounds. Resin 126, also known as benjoin djaoui, is included for antimicrobial properties and enhances hair shine. Musk ambrette 128 provides a natural fragrance while acting as a mild conditioning agent. Prunus mahaleb 130 contains phenolic compounds that support scalp health, while clove 132 is included for its antifungal properties and ability to improve blood circulation to the scalp.

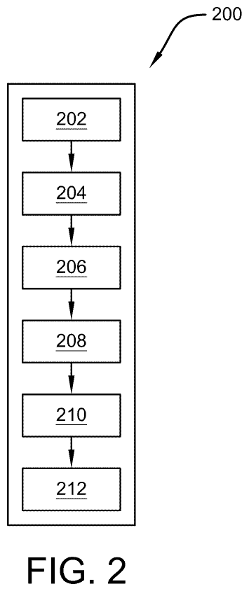

The present invention is also comprised of a method 200 of making the topical hair growth oil compound 100, as seen in FIG. 2. In a preferred embodiment, the method 200 produces 64 ounces of compound 110. First, 32 ounces of olive oil 102, 20 ounces of coconut oil 104, and 10 ounces of vitamin E oil 106 are combined to create an oil mixture [Step 202]. Then, two tablespoons of dried rosemary herb 108 and two tablespoons of dried thyme herb 110 are added to the oil mixture to allow the release of their active compounds [Step 204]. Next, three tablespoons of chebe powder 120 are introduced into the oil mixture, wherein the chebe powder 120 is comprised of lavender croton 122, vegetable oil 124, resin 126, musk ambrette 128, prunus mahaleb 130, and clove 132 [Step 206]. The combined mixture is then sealed within a container and is allowed to infuse at room temperature for preferably, but not limited to, seven days [Step 208]. Once the infusion period is complete, the container is shaken to redistribute settled components [Step 210]. Finally, the infused oil is filtered as necessary to remove excess herbal particulates and transferred into individual application bottles for user convenience [Step 212]. While the preferred embodiment produces a 64 ounces of compound 100, the total amount and ratio of ingredients may be adjusted based on specific formulation needs, user preferences, or production requirements.

To optimize hair growth stimulation and scalp nourishment, additional essential oils and bioactive ingredients may be incorporated into the compound 100. For example, peppermint oil 140 can be added to increase blood circulation to the hair follicles, enhancing nutrient delivery and stimulating follicular activity. Tea tree oil 142 can be incorporated for antimicrobial effects, reducing scalp buildup and preventing dandruff. Jamaican black castor oil 144 can be used to strengthen hair roots, improve thickness, and locks in moisture. Silk protein 146 or collagen 148 can be included to reinforce the hair structure, improving elasticity and reducing breakage.

The topical hair growth oil compound can also be tailored to meet specific hair care needs through specialized blends. A strengthening blend incorporates increased levels of silk protein 146 and collagen 148 to support fragile or damaged hair. A moisture lock blend includes hyaluronic acid 150 or aloe vera 152 to enhance scalp hydration and moisture retention. An ultra-growth blend integrates biotin 154 and caffeine 156 to stimulate hair follicles and promote accelerated regrowth. Each formulation variation is designed to cater to different hair concerns, ensuring optimal nourishment, strengthening, and regrowth support.

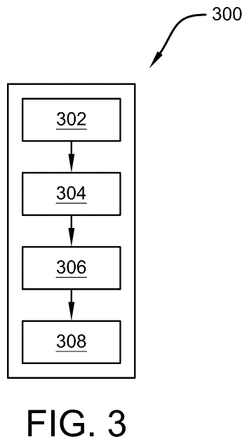

The present invention is also comprised of a method 300 of using the topical hair growth oil compound 100, as seen in FIG. 3. First, an application bottle containing the topical hair growth oil compound is provided [Step 302]. Then, a measured amount of the oil compound is dispensed onto the fingertips or directly onto the scalp [Step 304]. Next, the oil compound is gently massaged into the scalp using circular motions to ensure even distribution and stimulate blood circulation [Step 306]. Finally, the oil compound is left on the scalp for an extended period, such as, but not limited to, overnight or for several hours, before being washed out or left in as a leave-in treatment, depending on user preference [Step 308].

The topical hair growth oil compound 100 is designed to promote hair health through a blend of carefully selected natural oils, botanical extracts, and bioactive ingredients. The infusion process ensures the effective release of nutrients, while optional enhancements and specialized formulations provide adaptability for various hair care needs. The described formulation, along with its variations, offers a comprehensive approach to hair strengthening, hydration, and growth stimulation.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein, “topical hair growth oil compound” and “compound” are interchangeable and refer to the topical hair growth oil compound 100 of the present invention.

Notwithstanding the foregoing, the topical hair growth oil compound 100 of the present invention and its various components can be of any suitable size and configuration as is known in the art without affecting the overall concept of the invention, provided that they accomplish the above-stated objectives. One of ordinary skill in the art will appreciate that the size, configuration, and material of the topical hair growth oil compound 100 as shown in the FIGS. are for illustrative purposes only, and that many other sizes and shapes of the topical hair growth oil compound 100 are well within the scope of the present disclosure. Although the dimensions of the topical hair growth oil compound 100 are important design parameters for user convenience, the topical hair growth oil compound 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A topical hair growth oil compound comprising:
an olive oil;
a coconut oil;
a vitamin E oil;
a dried rosemary herb;
a dried thyme herb; and
a chebe powder.

2. The topical hair growth oil compound of claim 1, wherein the chebe powder is comprised of a lavender croton, a vegetable oil, a resin, a musk ambrette, a prunus mahaleb, and a clove.

3. The topical hair growth oil compound of claim 1 further comprised of a peppermint oil.

4. The topical hair growth oil compound of claim 1 further comprised of a tea tree oil.

5. The topical hair growth oil compound of claim 1 further comprised of a castor oil.

6. The topical hair growth oil compound of claim 1 further comprised of a silk protein.

7. The topical hair growth oil compound of claim 1 further comprised of a collagen.

8. The topical hair growth oil compound of claim 1 further comprised of an aloe vera.

9. The topical hair growth oil compound of claim 1 further comprised of a hyaluronic acid.

10. The topical hair growth oil compound of claim 1 further comprised of a biotin.

11. The topical hair growth oil compound of claim 1 further comprised of a caffeine.

12. A method of making a topical hair growth oil compound, the method comprising the following steps:
combining an olive oil, a coconut oil, and a vitamin E oil to form an oil mixture;
adding a dried rosemary herb and a dried thyme herb to the oil mixture;
adding a chebe powder into the oil mixture;
sealing the mixture and allowing the mixture to infuse at room temperature for a predetermined period;
shaking the mixture to redistribute settled components; and
filtering the mixture to remove an excess herbal particulate.

13. The topical hair growth oil compound of claim 12, wherein the predetermined period is comprised of seven days.

14. The topical hair growth oil compound of claim 12 further comprised of adding a peppermint oil.

15. The topical hair growth oil compound of claim 12 further comprised of adding a tea tree oil.

16. The topical hair growth oil compound of claim 12 further comprised of adding a castor oil.

17. The topical hair growth oil compound of claim 12 further comprised of adding a silk protein or a collagen.

18. The topical hair growth oil compound of claim 12 further comprised of adding an aloe vera or a hyaluronic acid.

19. The topical hair growth oil compound of claim 12 further comprised of adding a biotin or a caffeine.

20. A method of using a topical hair growth oil compound, the method comprising the following steps:
providing a topical hair growth oil compound comprised of an olive oil, a coconut oil, a vitamin E oil, a dried rosemary herb, a dried thyme herb, and a chebe powder;
dispensing the topical hair growth oil compound onto a fingertip or directly onto a scalp;
massaging the topical hair growth oil compound into the scalp using a circular motion to ensure even distribution and stimulate blood circulation; and
leaving the topical hair growth oil compound on the scalp for a period before washing the topical hair growth oil compound off of the scalp.
